(19) United States
(12) Patent Application Publication
(10) Pub. No.: US 2026/0245680 A1
(43) Pub. Date: Aug. 20, 2026

(54) SYSTEMS FOR VALIDATING ELECTRICALLY EVOKED COMPOUND ACTION POTENTIAL (ECAP) RECORDINGS

(71) Applicant: Ohio State Innovation Foundation, Columbus, OH (US)

(72) Inventors: Jeffrey SKIDMORE, Apex, NC (US); Shuman HE, Columbus, OH (US)

(21) Appl. No.: 19/164,488

(22) PCT Filed: Mar. 15, 2024
(86) PCT No.: PCT/US2024/020178

§ 371 Date: Sep. 11, 2025

Related U.S. Application Data
(60) Provisional application No. 63/490,588, filed on Mar. 16, 2023.

(51) Int. Cl. G16H 10/60; A61B 5/388

(52) U.S. Cl. CPC G16H 10/60; A61B 5/388

(57) ABSTRACT

Disclosed herein are systems, methods, and computer-program products for automatically detecting the presence of neural responses and incomplete masking in recordings of the electrically evoked compound action potential (eCAP).

### CROSS REFERENCE TO RELATED APPLICATION

This application claims priority to and benefit of U.S. Provisional Patent Application Ser. No. 63/490,588 filed Mar. 16, 2023, which is fully incorporated by reference and made a part hereof.

### GOVERNMENT SUPPORT CLAUSE

This invention was made with government support under grant/contract number DC016038 awarded by the National Institutes of Health. The government has certain rights in the invention.

### BACKGROUND

As shown in FIG. 1, cochlear implants convert sounds (picked up by a microphone) into electrical representations (in the sound processor) which are transmitted through the head and stimulate nerves surrounding the cochlea. Cochlear implants bypass the normal hearing pathway (i.e. middle ear bones and inner ear hair cells), which is usually not functional when somebody is deaf. However, cochlear implants still rely on the cochlear nerve to be functional in order to carry the signal to the brain for further processing and interpretation (as shown in FIG. 2).

In humans, there is no way to directly evaluate how well the cochlear nerve functions. However, as shown in FIG. 3, user defined stimuli can be sent through one electrode to stimulate the surrounding neurons. The neurons then respond, and the electrical response can be recorded by a neighboring electrode. The neural response is called the electrically-evoked compound action potential (eCAP). The eCAP response can be characterized as an indirect measure of neural function.

There are clinical applications of detected eCAPs. See, for example, “SYSTEMS AND METHODS FOR DETECTING THE PRESENCE OF ELECTRICALLY EVOKED COMPOUND ACTION POTENTIALS (eCAPs), ESTIMATING SURVIVAL OF AUDITORY NERVE FIBERS, AND DETERMINING EFFECTS OF ADVANCED AGE ON THE ELECTRODE-NEURON INTERFACE IN COCHLEAR IMPLANT USERS,” PCT/US2021/033604, published as WO 2021/236059 on Nov. 25, 2021, which is fully incorporated by reference and made a part hereof.

However, a challenge for any clinical application of eCAP technology is that the voltage recorded from the stimulation (i.e., stimulation artifact) is 1 to 2 orders of magnitude bigger than the voltage recorded from the neural response. Therefore, the stimulation artifact must be removed from the recording to view and isolate the neural response. Recording artifacts are also present in the eCAP measurements but are much smaller than the stimulation artifacts.

Previous attempts at resolving this challenge include the alternating polarity method, which assumes cathodic leading and anodic leading pulses result in the same neural response (amplitude and latency), which has repeatedly been shown to not be true. Other methods include forward masking methods. Single pulse forward masking assumes that all neurons are in refractory state after a first pulse. This is sometimes true but cannot be guaranteed in every instance. This method requires an expert to determine for each recording individually. Pulse train forward masking assumes the stimulation artifact from a single pulse is the same as the stimulation artifact from the last pulse of a pulse train, which is not true.

The “gold standard” method (i.e., two pulse forward masking) for removing stimulation artifacts from recordings of the electrically evoked compound action potential (eCAP) relies on assumptions that are frequently not met. However, the validity of the assumptions cannot be verified, except by expert researchers who access data not readily available.

The above leads to several challenges as invalid results are often assumed/used as valid because most people do not know how to check the assumptions, and the eCAP recordings may not be clinically viable.

Therefore, systems and methods are desired that overcome challenges in the art, some of which are described above. More specifically, there is a need for systems and methods to automatically check the validity of the two pulse forward masking paradigm and/or automatically detect if a neural response exists in eCAP recordings.

### SUMMARY

Disclosed and described herein are systems and methods to address the above-described challenges. In particular, systems and methods are disclosed to automatically a) detect the presence of electrically evoked compound action potentials (eCAPs) and b) determine the validity of the “gold standard” (i.e., two pulse forward masking) in reducing stimulation artifacts in eCAP recordings. The disclosed systems and methods address challenges preventing the application of eCAPs in clinical care of the hearing impaired. It will allow clinicians—as opposed to only a few expert researchers—to understand how the auditory nerve encodes electrically delivered auditory information. It will also introduce precision medicine for individual cochlear implant patients by allowing clinicians to make changes to the patient's cochlear implant settings based on how the patient's auditory nerve responds to electrical stimulation.

Systems and methods are disclosed herein for removing stimulation artifacts from electrical recordings that contain both the stimulation artifact and the neural response. One embodiment of the method comprises Systems for implementing this method are also described herein.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 is an image illustrating an example of a cochlear implant.

FIG. 2 is an image illustrating how cochlear implants still rely on the cochlear nerve to be functional in order to carry the signal to the brain for further processing and interpretation.

FIG. 3 is an illustration of how user defined stimuli can be sent through one electrode of a cochlear implant to stimulate the surrounding neurons, with the compound neural response (i.e., eCAP) being recorded by a different electrode of the cochlear implant.

FIGS. 4A-4C illustrate examples of conventional methods of removing stimulation artifacts from recorded eCAP traces.

FIG. 5A is a flowchart that discloses an exemplary method to remove stimulation artifacts from physiological recordings.

FIG. 5B illustrates that peaks in an electrical physiological trace (e.g., an A trace or a B trace) can be identified by finding min or max values in local regions of the trace, or by finding where the first derivative of the trace is equal to zero.

FIG. 5C illustrates an example where peaks are not readily identified in the recording trace (A trace—top panel), but a clear inflection is observed in the derivative trace (A′ trace—bottom panel), thus a neural response is present.

FIGS. 5D-5F are examples of detecting the presence or absence of eCAPs from various electrical physiological traces (e.g., A or B traces).

FIG. 6A is a flowchart that discloses an exemplary method to automatically detect incomplete masking in electrical physiological recordings (e.g., recordings of the electrically evoked compound action potential (eCAP)). FIG. 6B illustrates an example where peaks are readily identified in the recording trace (B trace—top panel), and a clear inflection is observed in the derivative trace (B′ trace—bottom panel), which indicates that incomplete masking is occurring.

FIG. 6C is another example of invalid forward masking as FIG. 6C shows both, peaks in the B trace (top frame), and an inflection point in the derivative trace (B′ trace—bottom panel).

FIG. 6D is yet another example of invalid forward masking as FIG. 6D shows both, peaks in the B trace (top frame), and an inflection point in the derivative trace (B′ trace—bottom panel).

FIG. 6E is an example of valid forward masking (i.e., assumption that all the neurons are placed into a refractory state cannot be proved to be invalid and therefore is likely to be valid), as there are no peaks in the B trace (top panel), and there are no inflection points as revealed in the first derivative (bottom panel).

FIGS. 7A and 7B illustrate that a rational function (e.g., hyperbola) best characterizes stimulation and recording artifacts (i.e., B-C+D trace of a fully masked stimulation).

FIGS. 8A and 8B illustrate that for the various A traces, with varying current stimulation levels as shown in FIG. 7A, the B-C+D trace of a fully masked stimulation for each of the A traces in FIG. 7A, as shown in FIG. 7B, can be modeled as a hyperbola, which is a specific form of a rational function.

FIG. 9 illustrates an exemplary computing device that can be used according to embodiments described herein.

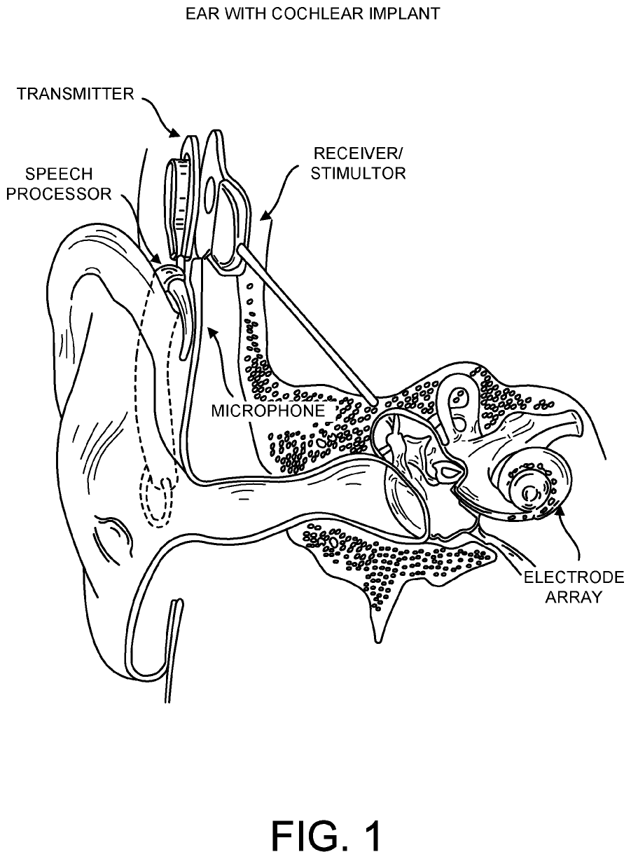

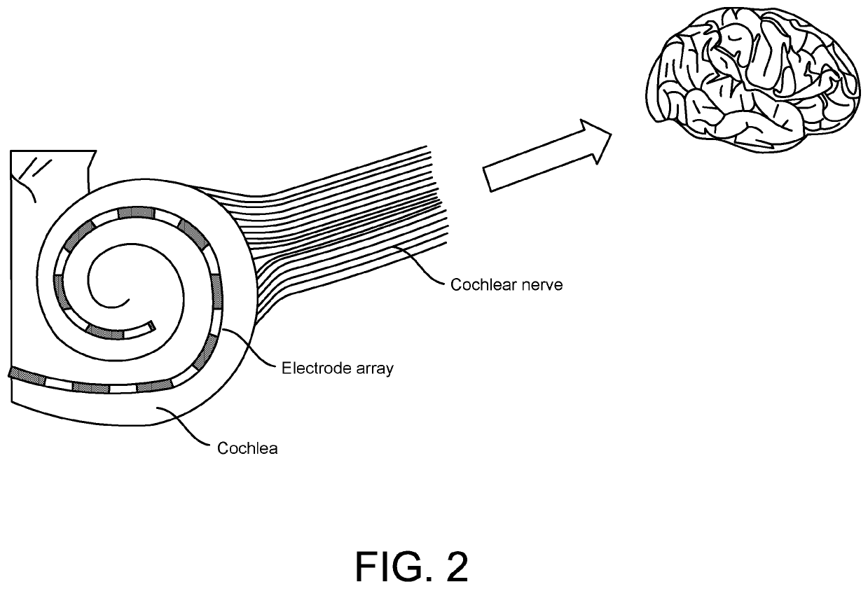

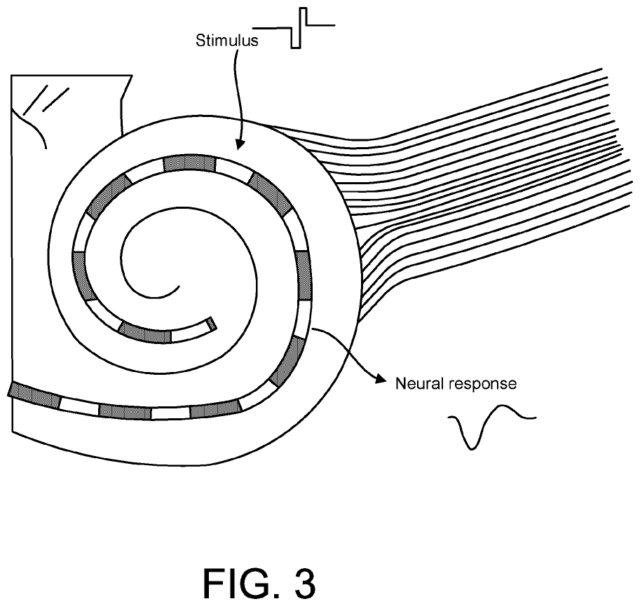

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

What is desired is systems and methods to reliably DETECT THE PRESENCE OF NEURAL RESPONSES AND INCOMPLETE MASKING IN RECORDINGS OF THE ELECTRICALLY EVOKED COMPOUND ACTION POTENTIAL (eCAP), which are used in cochlear implants, when using the 2-pulse forward masking method.

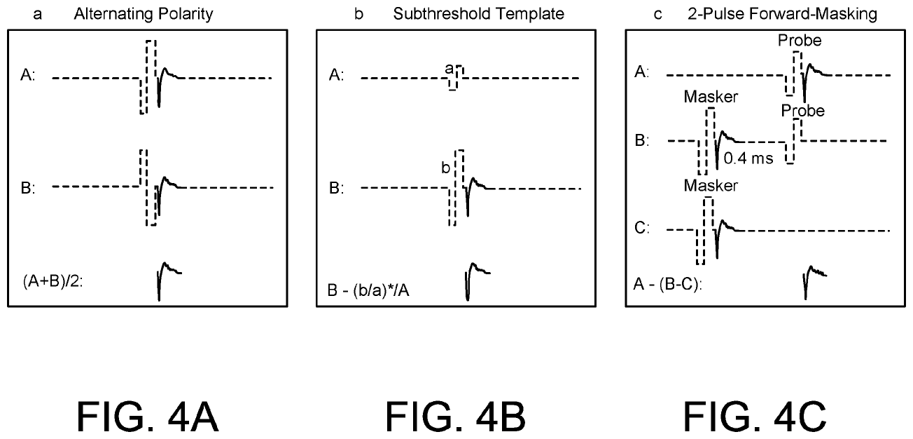

FIGS. 4A-4C illustrate examples of conventional methods of removing stimulation artifacts from recorded physiological traces. All of these conventional methods have limitations that make it challenging to use eCAP traces in clinical environments. FIG. 4A illustrates the alternating polarity method. In this method, a bi-phasic pulse is presented in two configurations—cathodic-leading (see trace “A” of FIG. 4A), and anodic-leading (see trace “B” of FIG. 4A). The theory behind this method is that the stimulation artifact will be removed when averaging the voltage recordings of these two simulations. However, this method relies on the assumptions that the stimulation artifacts from the two records are symmetric (i.e., cancel each other out) and that the eCAP response is the same for both stimulations. Both assumptions have repeatedly been shown to not be valid. FIG. 4B illustrates the subthreshold template method. In this method, a template of the stimulation artifact is obtained by measuring the voltage of a sub-threshold stimulation (see trace “A” of FIG. 4B) that does not produce a neural response. The voltage is then measured for the desired stimulation pulse at a higher stimulation level (see trace “B” of FIG. 4B). The subthreshold template is then scaled according to the difference in stimulation level and subtracted from the primary recording (see trace “B” of FIG. 4B). However, this method relies on the assumption that the stimulation artifact scales linearly with stimulation level which has repeatedly been shown to not be a valid assumption.

FIG. 4C illustrates the 2-pulse forward masking method, considered the conventional “gold standard” for removing stimulation artifacts from eCAP recordings. The idea is to create a “template” (i.e. voltage trace) that represents the stimulation artifact. This is done, in theory, by using a masker pulse that puts the neurons in a refractory state, so that there is not a neural response to the probe pulse (referred to hereafter as a “B” trace, as shown in FIG. 4C). By subtracting of the recording of the masker alone (referred to hereafter as a “C” trace, as shown in FIG. 4C), the artifact “template” is created. This template (B trace−C trace) can be subtracted from the recorded trace of interest (trace “A” of FIG. 4C), presumably leaving just the neural response (i.e., eCAP). However, this method fundamentally relies on the first pulse (masker) completely prohibiting any neural response in the second pulse (probe) by the masker fully putting all neurons in a refractory state. This assumption is only valid for some stimulation conditions as the neurons are affected by choice of masker stimulation level and the masker probe interval (MPI). Furthermore, conventional clinical software only shows the final calculated result A−(B−C), but the B trace recording must be analyzed to verify assumptions are valid. Therefore, as noted herein, the validity of the assumptions cannot be verified, except by expert researchers who access data not readily available. The above leads to several challenges, as invalid results are often assumed/used as valid because most people do not know how to check the assumptions, which leads to eCAP recordings that are not clinically viable.

Note: A “D” trace represents the recording artifact, but because it is much smaller than the stimulation artifact it cannot be shown on the scale of FIGS. 4A-4C, but ought to be considered as well. Therefore, D is included in the analyses that follow.

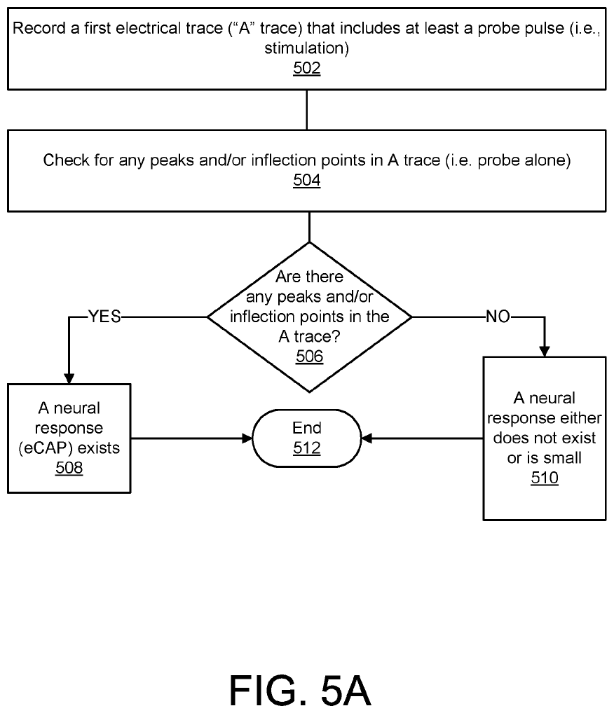

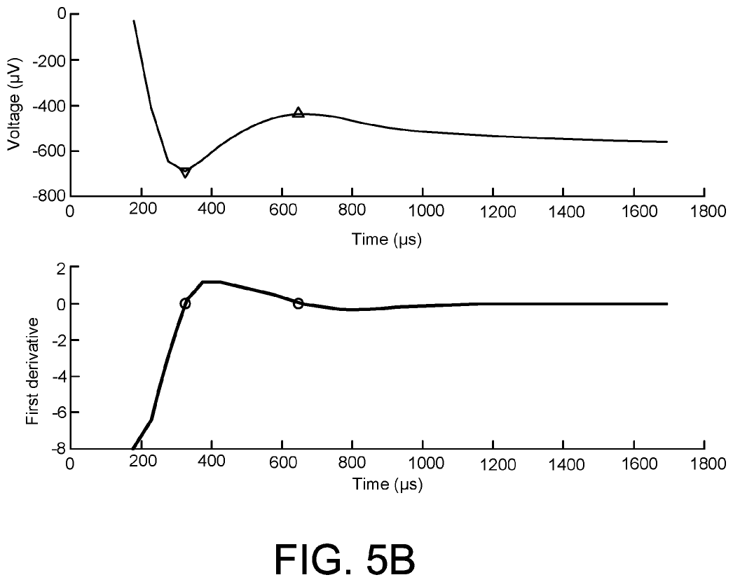

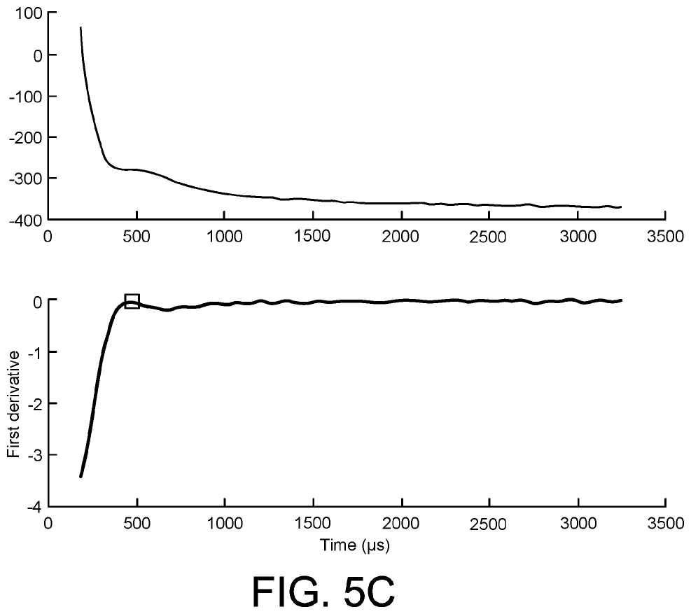

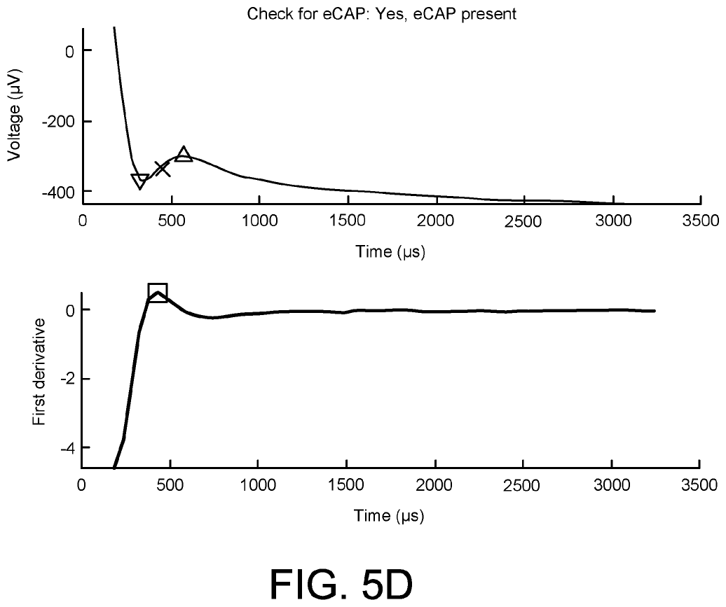

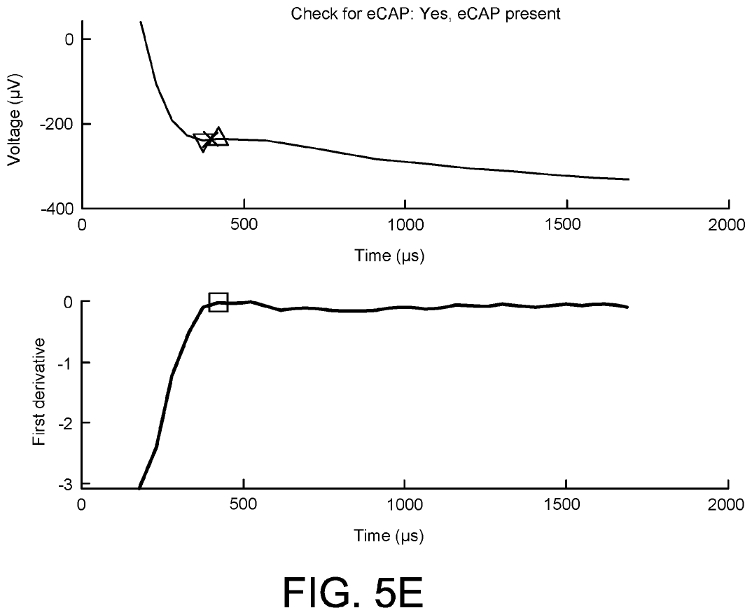

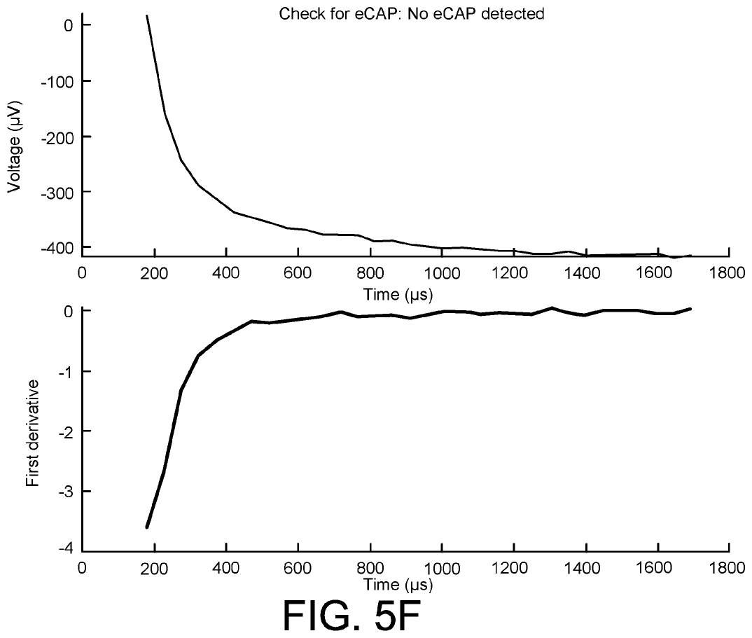

FIG. 5A is a flowchart that discloses an exemplary method to automatically detect the presence of neural responses (electrically evoked compound action potential (eCAPs)). As shown in FIG. 5A, the process begins at 502 by recording a first electrical trace (“A” trace) that includes at least the probe pulse (stimulation). In some instances, the A trace is recorded for a first period of time (e.g., for about 3200 microseconds (μs)).

At step 504, a check is made for any peaks and/or inflection points in the A trace (i.e., probe alone). As shown in FIG. 5B, peaks in the A trace can be identified by finding min or max values in local regions of the A trace, or by finding where the first derivative of the A trace is equal to zero. Checking for inflection points in the A trace comprises taking a first derivative of the A trace (referred to as an A′ trace) and identifying a peak in the A′ trace. FIG. 5C illustrates an example where peaks are not readily identified in the recording trace (A trace−top panel), but a clear inflection is observed in the derivative trace (A′ trace−bottom panel).

At step 506, if there are any peaks and/or inflection points in the A trace, then a neural response (eCAP) exists 508, and the process ends 512. If, at 506, there are no peaks and/or inflection points in the A trace, then the neural response either does not exist or is small 510, and the process ends 512.

FIGS. 5D-5F are examples of detecting the presence or absence of eCAPs from various A traces. FIG. 5D illustrates an example of detecting a presence of an eCAP as there are peaks in the A trace (top panel), and there is an inflection point in the first derivative (A′ trace−bottom panel). FIG. 5E illustrates another example of detecting a presence of an eCAP as there are peaks in the A trace (top panel), and there is an inflection point in the first derivative (A′ trace−bottom panel). FIG. 5F illustrates an example of detecting a lack of presence of an eCAP. As shown in FIG. 5F, there are no peaks in the A trace (top panel), and there is not an inflection point in the first derivative (A′ trace−bottom panel).

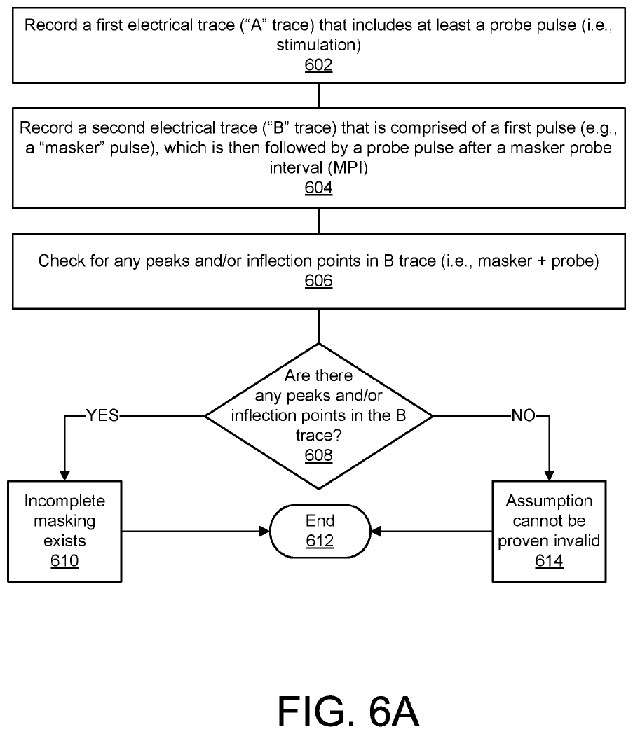

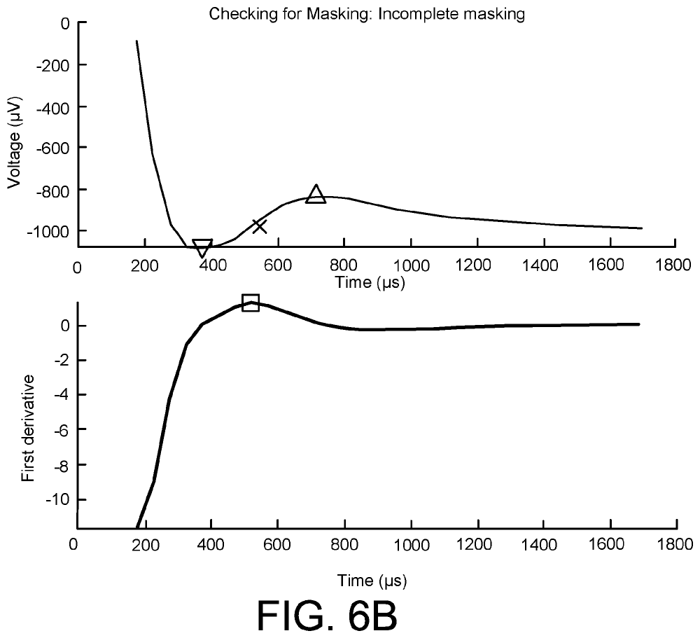

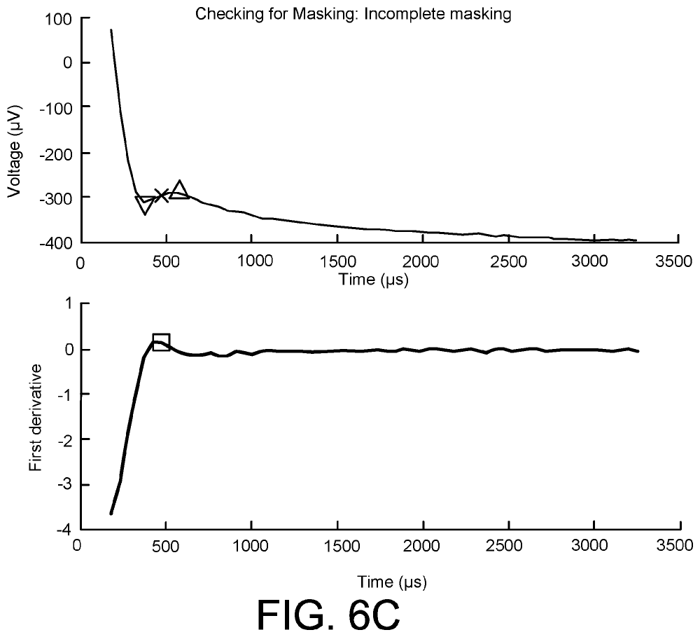

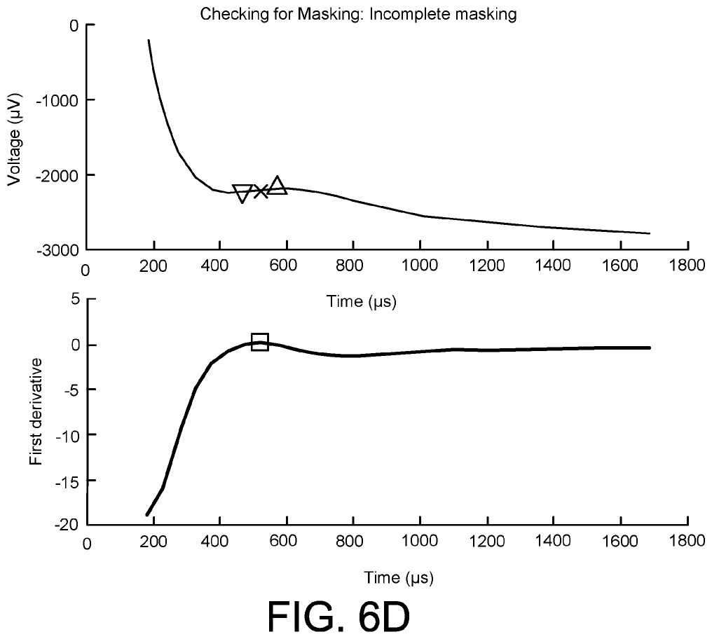

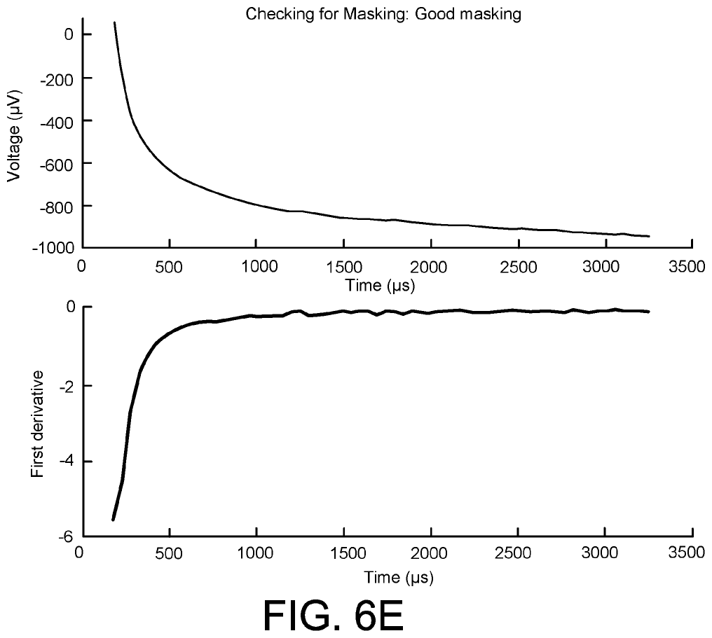

FIG. 6A is a flowchart that discloses an exemplary method to automatically detect incomplete masking in electrical physiological recordings (e.g., recordings of the electrically evoked compound action potential (eCAP)).

As shown in FIG. 6A, the process begins at 602 by recording a first electrical trace (“A” trace) that includes both the probe pulse and any resultant neural response. In some instances, the A trace is recorded for a first period of time (e.g., for about 3200 microseconds (μs)).

At step 604, a second electrical trace (“B” trace) is recorded. This trace is comprised of a first pulse (e.g., a “masker” pulse), which is then followed by a probe pulse after a masker probe interval (MPI). The masker pulse creates a resultant neural response. For the 2-pulse forward masking method to be effective, the first pulse (masker) should completely prohibit any neural response in the second pulse (probe) by putting all neurons in a refractory state. Thus, there should not be a neural response after the probe pulse.

With conventional 2-pulse forward masking, a third electrical trace (“C” trace) is recorded. This third electrical trace comprises the masker pulse and any resultant neural response. Optionally, a fourth trace (“D” trace) may be recorded, which is any recording artifact. The neural response is then found mathematically by subtracting (B trace−C trace), or B−(C+D) from the stimulation recorded trace of interest (A trace), presumably leaving just the neural response (i.e., eCAP).

At step 606, a check is made for any peaks and/or inflection points in the B trace (i.e., masker+probe). Peaks in the B trace can be identified by finding min or max values in local regions of the B trace, or by finding where the first derivative of the B trace is equal to zero. Checking for inflection points in the B trace comprises taking a first derivative of the B trace (referred to as a B′ trace) and identifying a peak in the B′ trace.

At step 608, if there are any peaks and/or inflection points in the B trace, then incomplete masking exists 610, and the process ends 612. As used herein, “incomplete masking” means that the first pulse (masker) did not completely prohibit any neural response in the second pulse (probe) by the masker fully putting all neurons in a refractory state at the time of the probe. If, at 608, there are no peaks and/or inflection points in the B trace, then the assumption that all the neurons are placed into a refractory state cannot be proved to be invalid and therefore is likely to be valid 614, and the process ends 612. FIG. 6B illustrates an example where peaks are readily identified in the recording trace (B trace−top panel), and a clear inflection is observed in the derivative trace (B′ trace−bottom panel), which indicates that there is incomplete masking occurring.

FIG. 6C is another example of invalid forward masking as FIG. 6C shows both, peaks in the B trace (top frame), and an inflection point in the derivative trace (B′ trace—bottom panel). FIG. 6D is yet another example of invalid forward masking as FIG. 6D shows both, peaks in the B trace (top frame), and an inflection point in the derivative trace (B′ trace−bottom panel). FIG. 6E is an example of valid forward masking (i.e., assumption that all the neurons are placed into a refractory state cannot be proved to be invalid and therefore is likely to be valid), as there are no peaks in the B trace (top panel), and there are no inflection points as revealed in the first derivative (bottom panel).

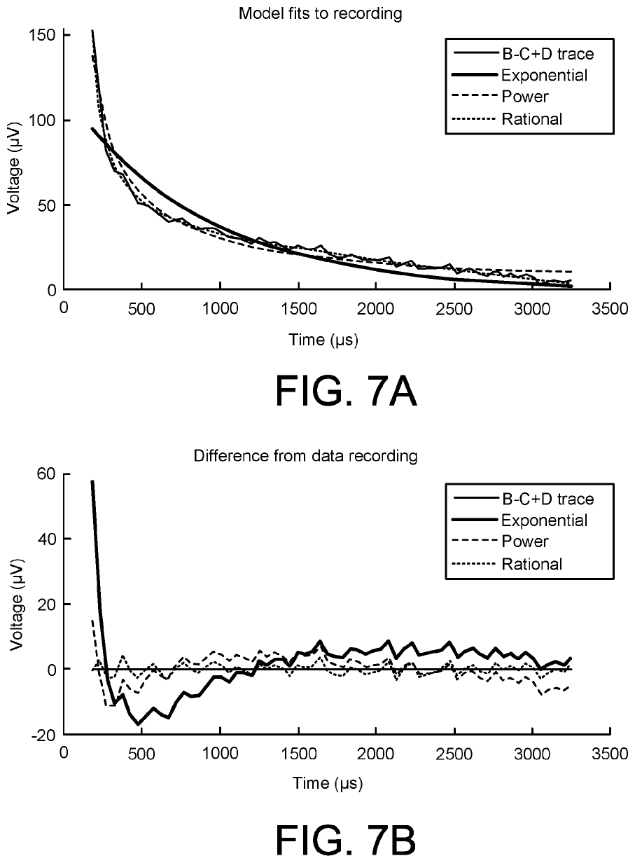

It is to be appreciated that the disclosed methods show that peaks and/or inflection points in A or B trace indicate that there is a neural response to the probe pulse. This is because stimulation artifacts can be modeled as being smooth rational functions, which validates that peaks and/or inflection points in A or B trace indicate neural response. this process is shown in greater detail with reference to FIGS. 7A and 7B, which illustrate that a hyperbola (i.e., rational function) best characterizes the artifacts (i.e., B−C+D trace of a fully masked stimulation). FIG. 7A illustrates the B−C+D trace of the fully masked stimulation and how an exponential function (i.e., y=ae^{bx}+ce^{dx}), a power function (i.e., y=ax^{b}+c) and the disclosed rational function fit to the B−C+D trace. The rational function in this instance is a hyperbola, which can be described by the following rational function:

$$y = \frac{{p}_{1} {x}^{2} + {p}_{2} x + {p}_{3}}{x + {q}_{1}}$$

It has the following asymptotes that match the recorded data:

$$Vertical asymptote : x = - {q}_{1}$$

$$Slanted asymptote : y = {p}_{1} x + {p}_{2} - {p}_{1} {q}_{1}$$

FIG. 7B illustrates the difference from the data recording for each of the exponential, power and rational functions as compared to a 0 line (no difference). As can be seen, the rational function tracks the recorded data much closer than either the exponential or power functions.

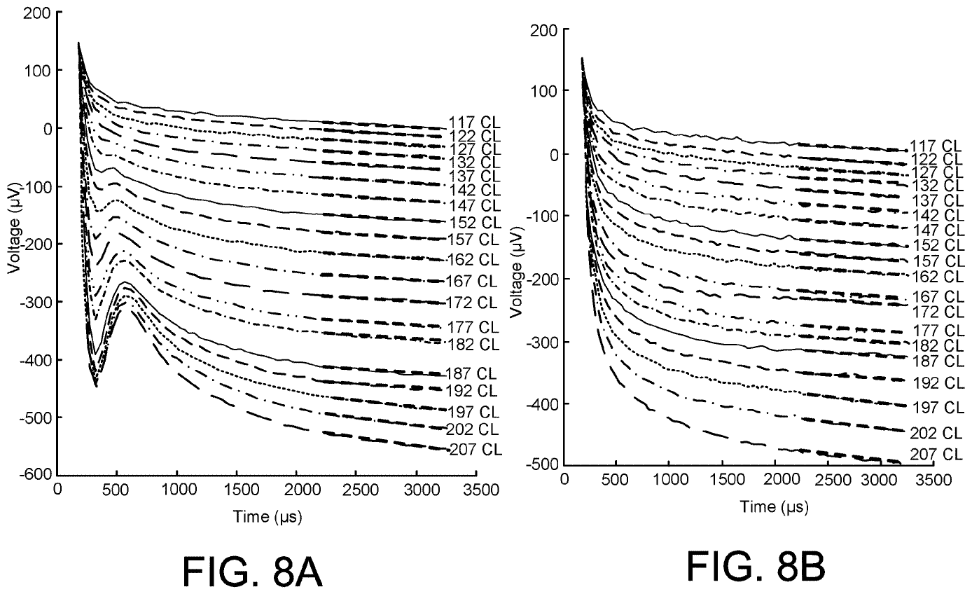

FIGS. 8A and 8B show that for the various A traces, with varying current stimulation levels as shown in FIG. 7A, the B−C+D trace of a fully masked stimulation for each of the A traces in FIG. 7A, as shown in FIG. 7B, can be modeled as a hyperbola, which is described by the rational function above. As can be seen in FIG. 7A, the A traces contain a visible neural response from about 300 μs to 800 μs at the higher stimulation levels, as well as the stimulation artifact throughout the entire recording. Each B−C+D trace (having been fully masked) of FIG. 7B shows a decaying artifact, significant non-zero slope, and normal residuals of “tail.” This demonstrates clearly that the stimulation artifact can be modeled as a hyperbola at all of the stimulation levels.

Non-limiting advantages of the disclosed embodiments include: 1) the enhanced clinical use of eCAPs as the disclosed embodiments provide automatic detection of the presence of eCAP (i.e. neural response) and if the result of the “Gold standard” (i.e., 2-pulse forward masking) is valid or not. Further, the results of the disclosed embodiments do not require an expert researcher to determine validity.

While the embodiments of this invention as described herein were developed for cochlear implants, embodiments also apply to any other implantable stimulation device that delivers electrical stimuli and are considered within the scope of this disclosure. Examples include: pacemakers; defibrillators; retinal stimulators; muscle stimulators; spinal cord stimulators; deep brain stimulators; and neural stimulators to treat urinary incontinence, sleep apnea, shoulder subluxation, and the like.

### I. COMPUTING ENVIRONMENT

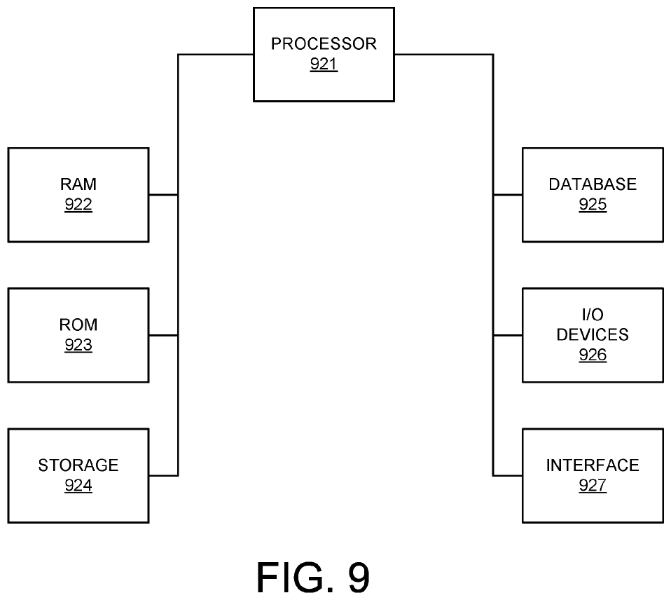

The above-described methods may be implemented on a computing system. The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for methods of removing stimulation artifacts from physiological recordings after single and multi-pulses. In one exemplary aspect, the units can comprise a computing device that comprises a processor 921 as illustrated in FIG. 9 and described below.

FIG. 9 illustrates an exemplary computer that can be used for automatically detecting the presence of neural responses and incomplete masking in recordings of the electrically evoked compound action potential (eCAP). As used herein, “computer” may include a plurality of computers. The computers may include one or more hardware components such as, for example, a processor 921, a random-access memory (RAM) module 922, a read-only memory (ROM) module 923, a storage 924, a database 925, one or more input/output (I/O) devices 926, and an interface 927. All of the hardware components listed above may not be necessary to practice the methods described herein. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 924 may include a software partition associated with one or more other hardware components. It is understood that the components listed above are exemplary only and not intended to be limiting.

Processor 921 may include one or more processors, each configured to execute instructions and process data to perform one or more functions associated with removing stimulation artifacts from physiological recordings after single and multi-pulses. Processor 921 may be communicatively coupled to RAM 922, ROM 923, storage 924, database 925, I/O devices 926, and interface 927. Processor 921 may be configured to execute sequences of computer program instructions to perform various processes. The computer program instructions may be loaded into RAM 922 for execution by processor 921.

RAM 922 and ROM 923 may each include one or more devices for storing information associated with operation of processor 921. For example, ROM 923 may include a memory device configured to access and store information associated with the computer, including information for identifying, initializing, and monitoring the operation of one or more components and subsystems. RAM 922 may include a memory device for storing data associated with one or more operations of processor 921. For example, ROM 923 may load instructions into RAM 922 for execution by processor 921.

Storage 924 may include any type of mass storage device configured to store information that processor 921 may need to perform processes consistent with the disclosed embodiments. For example, storage 924 may include one or more magnetic and/or optical disk devices, such as hard drives, CD-ROMs, DVD-ROMs, or any other type of mass media device.

Database 925 may include one or more software and/or hardware components that cooperate to store, organize, sort, filter, and/or arrange data used by the computer and/or processor 921. For example, database 925 may store raw data, as described herein and computer-executable instructions for removing stimulation artifacts from physiological recordings after single and multi-pulses. It is contemplated that database 925 may store additional and/or different information than that listed above.

I/O devices 926 may include one or more components configured to communicate information with a user associated with computer. For example, I/O devices may include a console with an integrated keyboard and mouse to allow a user to maintain a database of digital images, results of the analysis of the digital images, metrics, and the like. I/O devices 926 may also include a display including a graphical user interface (GUI) for outputting information on a monitor. I/O devices 926 may also include peripheral devices such as, for example, a printer for printing information associated with the computer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 927 may include one or more components configured to transmit and receive data via a communication network, such as the Internet, a local area network, a workstation peer-to-peer network, a direct link network, a wireless network, or any other suitable communication platform. For example, interface 927 may include one or more modulators, demodulators, multiplexers, demultiplexers, network communication devices, wireless devices, antennas, modems, and any other type of device configured to enable data communication via a communication network.

### II. CONCLUSION

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

The publications incorporated by reference include, but are not limited to, the following:
A. He, S., Teagle, H. F., & Buchman, C. A. (2017). The electrically evoked compound action potential: from laboratory to clinic. Frontiers in neuroscience, 11, 339.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method of automatically validating results of a 2-pulse forward masking method to identify a neural response comprising:
receiving a recorded A trace and/or a recorded B trace, wherein the recorded A trace and the recorded B trace are obtained from a patient as part of the 2-pulse forward masking method;
checking for any peaks and/or inflection points in the A trace and/or the B trace, wherein if there are any peaks and/or inflection points in the A trace and/or the B trace, then identify that a neural response is also included in the A trace and/or B trace, and if there are not any peaks and/or inflection points in the A trace and/or B trace, then identify that a neural response is not included in the A trace and/or B trace, said B trace comprising a masker pulse followed by a probe pulse after a masker probe interval (MPI); and
checking for any peaks and/or inflection points in the B trace, wherein if there are any peaks and/or inflection points in the B trace, then identify that incomplete masking exists for the 2-pulse forward masking method, and if there are not any peaks and/or inflection points in the B trace, then identify that assumptions that neurons of the patient are placed into a refractory state by the masker pulse cannot be proved to be invalid and therefore are likely to be valid.

2. The method of claim 1, wherein checking for any peaks in the A trace and/or the B trace comprises finding min or max values in local regions of the A trace and/or B trace.

3. The method of claim 1, wherein checking for any inflection points in the A trace and/or the B trace comprises taking a first derivative of the A trace and/or the B trace and finding one or more points where the first derivative equals zero.

4. A system for automatically validating results of a 2-pulse forward masking method to identify a neural response comprising:
a memory; and
a processor in communication with the memory, wherein the processor executes computer-executable instructions stored in the memory, said instructions causing the processor to:
receive a recorded A trace and/or a recorded B trace, wherein the recorded A trace and the recorded B trace are obtained from a patient as part of the 2-pulse forward masking method;
check for any peaks and/or inflection points in the A trace and/or the B trace, wherein if there are any peaks and/or inflection points in the A trace and/or the B trace, then identify that a neural response is also included in the A trace and/or B trace, and if there are not any peaks and/or inflection points in the A trace and/or B trace, then identify that a neural response is not included in the A trace and/or B trace, said B trace comprising a masker pulse followed by a probe pulse after a masker probe interval (MPI); and
check for any peaks and/or inflection points in the B trace, wherein if there are any peaks and/or inflection points in the B trace, then identify that incomplete masking exists for the 2-pulse forward masking method, and if there are not any peaks and/or inflection points in the B trace, then identify that assumptions that neurons of the patient are placed into a refractory state by the masker pulse cannot be proved to be invalid and therefore are likely to be valid.

5. The system of claim 4, wherein checking for any peaks in the A trace and/or the B trace comprises finding min or max values in local regions of the A trace and/or B trace.

6. The system of claim 4, wherein checking for any inflection points in the A trace and/or the B trace comprises taking a first derivative of the A trace and/or the B trace and finding one or more points where the first derivative equals zero.

7. A method to automatically detect the presence of neural responses (electrically evoked compound action potential (eCAPs)) from an electrical physiological recording comprising:
receiving a first recorded waveform trace obtained from a patient, wherein the first recorded waveform trace includes at least a probe pulse;
checking for any peaks and/or inflection points in the first recorded waveform trace; and
if there are any peaks and/or inflection points in the first recorded waveform trace, then identify that a neural response is also included in the first recorded waveform trace; else
if there are not any peaks and/or inflection points in the first recorded waveform trace, then identify that a neural response is not included in the first recorded waveform trace.

8. The method of claim 7, wherein the recorded waveform trace is an A trace or a B trace obtained as part of a 2-pulse forward masking method of detecting an electrically-evoked compound action potential (eCAP).

9. The method of claim 7, wherein checking for any peaks in the first recorded waveform trace comprises finding min or max values in local regions of the first recorded waveform trace.

10. The method of claim 7, wherein checking for any inflection points in the first recorded waveform trace comprises taking a first derivative of the first recorded waveform trace and finding one or more points where the first derivative equals zero.

11. A system for automatically detecting the presence of neural responses (electrically evoked compound action potential (eCAPs)) from an electrical physiological recording comprising:
a memory; and
a processor in communication with the memory, wherein the processor executes computer-executable instructions stored in the memory, said instructions causing the processor to:
receive a first recorded waveform trace obtained from a patient, wherein the first recorded waveform trace includes at least a probe pulse;
check for any peaks and/or inflection points in the first recorded waveform trace; and
if there are any peaks and/or inflection points in the first recorded waveform trace, then identify that a neural response is also included in the first recorded waveform trace; else
if there are not any peaks and/or inflection points in the first recorded waveform trace, then identify that a neural response is not included in the first recorded waveform trace.

12. The system of claim 11, wherein the recorded waveform trace is an A trace or a B trace obtained as part of a 2-pulse forward masking method of detecting an electrically-evoked compound action potential (eCAP).

13. The system of claim 11, wherein checking for any peaks in the first recorded waveform trace comprises finding min or max values in local regions of the first recorded waveform trace.

14. The system of claim 11, wherein checking for any inflection points in the first recorded waveform trace comprises taking a first derivative of the first recorded waveform trace and finding one or more points where the first derivative equals zero.

15. A method to automatically detect incomplete masking when detecting a presence of neural responses (electrically evoked compound action potential (eCAPs)) from an electrical physiological recording using a 2-pulse forward masking method comprising:
receiving a first recorded waveform trace (A trace) obtained from a patient, wherein the first recorded waveform trace includes at least a probe pulse;
receiving a second recorded waveform trace (B trace) obtained from the patient that is comprised of a masker pulse followed by a probe pulse after a masker probe interval (MPI); and
checking for any peaks and/or inflection points in the B trace, wherein if there are any peaks and/or inflection points in the B trace, then identify that incomplete masking exists for the 2-pulse forward masking method, and if there are not any peaks and/or inflection points in the B trace, then identify that assumptions that neurons of the patient are placed into a refractory state by the masker pulse cannot be proved to be invalid and therefore are likely to be valid.

16. The method of claim 15, wherein checking for any peaks in the B trace comprises finding min or max values in local regions of the B trace.

17. The method of claim 15, wherein checking for any inflection points in the B trace comprises taking a first derivative of the B trace and finding one or more points where the first derivative equals zero.

18. A system for automatically detecting incomplete masking when detecting a presence of neural responses (electrically evoked compound action potential (eCAPs)) from an electrical physiological recording using a 2-pulse forward masking method comprising:
a memory; and
a processor in communication with the memory, wherein the processor executes computer-executable instructions stored in the memory, said instructions causing the processor to:
receive a first recorded waveform trace (A trace) obtained from a patient, wherein the first recorded waveform trace includes at least a probe pulse;
receive a second recorded waveform trace (B trace) obtained from the patient that is comprised of a masker pulse followed by a probe pulse after a masker probe interval (MPI); and
check for any peaks and/or inflection points in the B trace, wherein if there are any peaks and/or inflection points in the B trace, then identify that incomplete masking exists for the 2-pulse forward masking method, and if there are not any peaks and/or inflection points in the B trace, then identify that assumptions that neurons of the patient are placed into a refractory state by the masker pulse cannot be proved to be invalid and therefore are likely to be valid.

19. The system of claim 18, wherein checking for any peaks in the B trace comprises finding min or max values in local regions of the B trace.

20. The system of claim 18, wherein checking for any inflection points in the B trace comprises taking a first derivative of the B trace and finding one or more points where the first derivative equals zero.

21. (canceled)

22. (canceled)

23. (canceled)

24. (canceled)

25. (canceled)

26. (canceled)

27. (canceled)

28. (canceled)

29. (canceled)

30. (canceled)
